(19) United States
(12) Patent Application Publication
(10) Pub. No.: US 2005/0129639 A1
(43) Pub. Date: Jun. 16, 2005

(54) Makeup composition comprising an emulsion

(76) Inventor: Eric Quemin, Joinville le Pont (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/982,924

(22) Filed: Nov. 8, 2004

Related U.S. Application Data
(60) Provisional application No. 60/520,639, filed on Nov. 18, 2003.

(30) Foreign Application Priority Data
Nov. 7, 2003 (FR) 03 50810

(51) Int. Cl. A61K 7/021; A61K 7/025; A61K 7/075; A61K 7/08

(52) U.S. Cl. 424/63; 424/64; 424/70.22

(57) ABSTRACT

The present disclosure relates to a stable makeup composition comprising: an emulsion comprising an oily phase dispersed in an aqueous phase, and solid particles dispersed in the aqueous phase of the emulsion such that the makeup composition is stable after storage for two months at 45° C., wherein the emulsion comprises a surfactant system comprising: at least one nonionic surfactant, and at least one ionic surfactant with a dynamic interface tension of less than or equal to 7 millinewtons/meter. Also disclosed herein is a process for making up keratin materials, such as the skin.

This application claims benefit of U.S. Provisional Application No. 60/520,639, filed Nov. 18, 2003.

The present disclosure relates to a makeup composition comprising an emulsion based on a system of surfactants and of dispersed solid particles. The present disclosure also relates to a process for preparing the composition and to its use for making up keratin materials, such as the skin.

Nanoemulsions are oil-in-water emulsions comprising oil globules of very fine particle size, that is to say that they have, for example, a number-average size of less than 500 nanometers (nm). They are frequently manufactured by mechanical fragmentation of an oily phase in an aqueous phase in the presence of surfactants. In the case of nanoemulsions, the very small size of the oily globules can be obtained for instance, by means of at least one treatment in a high-pressure homogenizer. The small size of the globules can provide them with cosmetically advantageous properties that distinguish them from standard emulsions: they may be translucent, or even transparent, and may have a novel texture. They may also convey active agents more efficiently.

Nanoemulsions comprising an amphiphilic lipid phase consisting of phospholipids, water and oil are known in the prior art. These emulsions can have the drawback of being unstable when stored at the traditional storage temperatures, for example, at a temperature ranging from 0° C. to 45° C. They can lead to yellow compositions and can produce rancid odors that develop after storage for a few days.

Nanoemulsions stabilized with a lamellar liquid crystal coating obtained by combining a hydrophilic surfactant and a lipophilic surfactant are also known. However, these combinations can be difficult to make. Furthermore, such nanoemulsions can have a waxy, film-forming feel that is frequently not pleasant for the user.

Patent Application No. EP-A-728 460 describes nanoemulsions containing two types of surfactants:
a nonionic surfactant selected from fatty esters of polyethylene glycol or of sorbitol; and
an ionic surfactant, namely potassium dicetyl phosphate.

Patent Application No. WO 02/056843 describes a translucent nanoemulsion comprising a ternary surfactant system, which may be used, for example, as a skin makeup composition. This nanoemulsion is obtained by high-pressure homogenization of the emulsion. However, the introduction of solid particles such as pigments, for instance iron oxides or titanium dioxides, or alternatively fillers, into the nanoemulsion can lead to several problems. For example, it is not possible to introduce the solid particles (such as pigments or fillers) during the manufacture of the nanoemulsion since these particles can damage the homogenization cell of the high-pressure homogenizer. Moreover, it can be necessary to grind the pigments as finely as possible to avoid the presence of lumps (or aggregates) of pigments in the composition, because these lumps of pigments can be detrimental to the production of good color, i.e., developed color, of the makeup. However, the grinding technique is not really compatible with the nanoemulsion since the nanoemulsion can be totally destroyed by the mechanical effects of grinding: it can be, therefore, impossible to grind the pigments with the nanoemulsion. Moreover, the direct introduction of the pigments or fillers into the nanoemulsion can cause the viscosity of the nanoemulsion to decrease and the pigments and/or fillers can sediment out in the composition: the solid particles (pigments or fillers) would then no longer be homogeneously distributed in the composition; the composition could therefore be unstable and would thus be unsuitable as a makeup product since it would not allow a uniform makeup result to be obtained.

There is thus a need to be able to introduce pigments or fillers into an emulsion, such as a nanoemulsion, in a satisfactory manner.

Accordingly, another aspect of the present disclosure is to provide a makeup composition comprising an emulsion, such as a nanoemulsion, and uniformly distributed solid particles (for instance pigments and fillers). Still another aspect of the present disclosure is also to provide a pigmented makeup composition comprising an emulsion, such as a nanoemulsion, which is stable during and after storage, for example, after storage for two months at a temperature less than or equal to 45° C.

The present inventors have discovered that it is possible to obtain a stable makeup composition by introducing the solid particles (such as pigments and fillers) predispersed in water with the aid of particular ionic surfactants, after the formation of the emulsion, for instance, the nanoemulsion.

For example, one embodiment of the present disclosure relates to a makeup composition comprising:
an emulsion comprising an oily phase dispersed in an aqueous phase, and solid particles dispersed in the aqueous phase of the emulsion such that the makeup composition is stable after storage for two months at a temperature less than or equal to 45° C.,
   wherein the emulsion comprises oil globules with a number-average size of less than or equal to 500 nm and in that it comprises a surfactant system comprising:
   at least one nonionic surfactant, and
   at least one ionic surfactant with a dynamic interface tension of less than or equal to 7 millinewtons/meter.

Another aspect of the present disclosure relates to a makeup composition comprising:
an emulsion comprising an oily phase dispersed in an aqueous phase and solid particles dispersed in the aqueous phase of the emulsion and having a number-average size of less than or equal to 10 μm, and
   wherein the emulsion comprises oil globules with a number-average size of less than or equal to 500 nm and in that it comprises a surfactant system comprising:
   at least one nonionic surfactant, and
   at least one ionic surfactant with a dynamic interface tension of less than or equal to 7 millinewtons/meter.

The present disclosure also relates to a makeup composition comprising:
an emulsion comprising an oily phase dispersed in an aqueous phase and solid particles dispersed in the aqueous phase of the emulsion such that the makeup composition is stable after storage for two months at a temperature less than or equal to 45° C.,
   wherein the emulsion comprises a surfactant system comprising:
   at least one nonionic surfactant, and
   at least one ionic surfactant with a dynamic interface tension of less than or equal to 7 millinewtons/meter, and
   wherein the solid particles comprise at least one pigment chosen from iron oxides, zinc oxides, zirconium oxides, cerium oxides, mica coated with titanium dioxide, mica coated with bismuth oxychloride, titanium mica coated with iron oxide, titanium mica coated with ferric blue, titanium mica coated with chromium oxide, and organic pigments.

Still another aspect of the present disclosure is a makeup composition comprising:
an emulsion comprising an oily phase dispersed in an aqueous phase, and solid particles dispersed in the aqueous phase of the emulsion and having a number-average size of less than or equal to 10 μm,
   wherein the emulsion comprises a surfactant system comprising:
   at least one nonionic surfactant, and
   at least one ionic surfactant with a dynamic interface tension of less than or equal to 7 millinewtons/meter, and
   wherein the solid particles comprise at least one pigment chosen from iron oxides, zinc oxides, zirconium oxides, cerium oxides, mica coated with titanium dioxide, mica coated with bismuth oxychloride, titanium mica coated with iron oxide, titanium mica coated with ferric blue, titanium mica coated with chromium oxide, and organic pigments.

The disclosure still further relates to a makeup composition comprising:
an emulsion comprising an oily phase dispersed in an aqueous phase, and solid particles dispersed in the aqueous phase of the emulsion such that the makeup composition is stable after storage for two months at a temperature less than or equal to 45° C.,
   wherein the emulsion comprises a surfactant system comprising:
   at least one nonionic surfactant, and
   at least one ionic surfactant with a dynamic interface tension of less than or equal to 7 millinewtons/meter, and
   wherein the oily phase is present in an amount of at least 20% by weight, relative to the total weight of the composition.

Another aspect of the present disclosure is a makeup composition comprising:
an emulsion comprising an oily phase dispersed in an aqueous phase, and solid particles dispersed in the aqueous phase of the emulsion and having a number-average size of less than or equal to 10 μm,
   wherein the emulsion comprises a surfactant system comprising:
   at least one nonionic surfactant, and
   at least one ionic surfactant with a dynamic interface tension of less than or equal to 7 millinewtons/meter, and
wherein the oily phase is present in an amount of at least 20% by weight, relative to the total weight of the composition.

Still another aspect of the present disclosure is a makeup composition comprising:
an emulsion comprising an oily phase dispersed in an aqueous phase, solid particles dispersed in the aqueous phase of the emulsion such that the makeup composition is stable after storage for two months at a temperature less than or equal to 45° C., and a nonionic aqueous thickener chosen from associative polyurethanes,
   wherein the emulsion comprises a surfactant system comprising:
   at least one nonionic surfactant, and
   at least one ionic surfactant with a dynamic interface tension of less than or equal to 7 millinewtons/meter.

Yet another aspect of the present disclosure is a makeup composition comprising:
an emulsion comprising an oily phase dispersed in an aqueous phase, solid particles dispersed in the aqueous phase of the emulsion and having a number-average size of less than or equal to 10 μm, and a nonionic aqueous thickener chosen from associative polyurethanes,
   wherein the emulsion comprises a surfactant system comprising:
   at least one nonionic surfactant, and
   at least one ionic surfactant with a dynamic interface tension of less than or equal to 7 millinewtons/meter.

An additional aspect of the present disclosure is a makeup composition comprising:
an emulsion comprising an oily phase dispersed in an aqueous phase, and solid particles dispersed in the aqueous phase of the emulsion such that the makeup composition is stable after storage for two months at a temperature less than or equal to 45° C.,
   wherein the emulsion comprises a surfactant system comprising:
   at least one nonionic surfactant comprising at least one ethoxylated fatty ester comprising from 10 to 80 ethylene oxide units and at least one fatty acid ester of sorbitan, and
   at least one ionic surfactant with a dynamic interface tension of less than or equal to 7 millinewtons/meter.

Still further, another aspect of the present disclosure is a makeup composition comprising:
an emulsion comprising an oily phase dispersed in an aqueous phase, and solid particles dispersed in the aqueous phase of the emulsion and having a number-average size of less than or equal to 10 μm,
   wherein the emulsion comprises a surfactant system comprising:
   at least one nonionic surfactant comprising at least one ethoxylated fatty ester comprising from 10 to 80 ethylene oxide units and at least one fatty acid ester of sorbitan, and
   at least one ionic surfactant with a dynamic interface tension of less than or equal to 7 millinewtons/meter.

A further additional aspect of the present disclosure is a cosmetic process for making up keratin materials, such as the skin, comprising applying to the keratin materials, such as the skin, a makeup composition comprising: an emulsion comprising an oily phase dispersed in an aqueous phase, and solid particles dispersed in the aqueous phase of the emulsion, wherein the emulsion comprises a surfactant system comprising
at least one nonionic surfactant, and
at least one ionic surfactant with a dynamic interface tension of less than or equal to 7 millinewtons/meter, and
wherein the solid particles dispersed in the aqueous phase of the emulsion are such that the makeup composition is stable after storage for two months at a temperature less than or equal to 45° C., and/or the solid particles have a number-average size of less than or equal to 10 μm.

Another aspect of the present disclosure is also a process for making up keratin materials, such as the skin, comprising the application to the skin of a composition as defined above.

The term “stable composition” means a composition that is stable after storage for two months at a temperature less than or equal to 45° C., for example, stable after storage for two months at a temperature less than or equal to 25° C., such as stable after storage for two months at 4° C.

For the purposes of the present disclosure, a stable composition is a composition that has a homogeneous dispersion of pigments, i.e. a composition that does not comprise aggregates of pigments that are visible to the naked eye when the makeup composition is applied to keratin materials, such as the skin. The makeup result should have a uniform color without leaving marks or trails of color. Further, the pigments can be fully distributed in the composition, thus making it possible to obtain a makeup product that has a uniform color when it is applied to keratin materials, such as the skin. Moreover, the pigments can be incorporated into the composition while at the same time conserving the cosmetic properties of the emulsion, such as the nanoemulsion, and accordingly, the composition can have, for example, good skin moisturizing properties.

The emulsion can comprise, for example, oil globules with a number-average size of less than or equal to 1 μm, for instance, ranging from 500 nm to 1 μm, such as less than or equal to 500 nm, for example, ranging from 50 nm to 500 nm, such as less than or equal to 300 nm, for instance, ranging from 50 nm to 300 nm, such as less than or equal to 200 nm, for example, ranging from 50 nm to 200 nm and ranging from 50 to 150 nm. The globule size may be measured, for example, using a Brookhaven BI 90 machine and determined according to the known method of “Quasi-elastic light scattering.” Without being bound by theory, it is believed that reducing the globule size makes it possible to promote the concentration of the active agents in the surface layers of the skin (vehicle effect).

The surfactant system that may be used in the emulsion of the according to the present disclosure comprises, as indicated above, at least one nonionic surfactant.

The at least one nonionic surfactant may be present in an amount ranging from 1% to 10% by weight, for example, ranging from 2% to 6% by weight, relative to the total weight of the composition.

The at least one nonionic surfactant may be chosen from ethoxylated fatty esters comprising 8 to 100 ethylene oxide units and fatty acid esters of sorbitan, and mixtures thereof. The esters of the at least one nonionic surfactant can comprise a fatty chain comprising from 16 to 22 carbon atoms. The fatty chain of the esters may be chosen from, for instance, stearyl, behenyl, arachidyl, palmityl and cetyl units, and mixtures thereof. In one embodiment of the present disclosure, fatty chain of the esters is cetearyl.

The number of ethylene oxide units may range from 8 to 100, for instance from 10 to 80, such as from 20 to 60. According to one embodiment of the present disclosure, this number is 40.

As an example of an ethoxylated fatty ester comprising 40 ethylene oxide units, non-limiting mention may be made of the stearic acid ester comprising 40 ethylene oxide units, such as the product sold under the name Myrj 52 (polyethylene glycol 40 EO stearate; CTFA name: PEG-40 stearate) by the company Uniqema.

A non-limiting example of a fatty acid ester of sorbitan that may be mentioned is sorbitan tristearate.

The at least one nonionic surfactant can be, for example, a mixture comprising at least one ethoxylated fatty ester comprising from 8 to 100 ethylene oxide units, such as from 10 to 80 ethylene oxide units, and at least one fatty acid ester of sorbitan.

By way of further non-limiting example, the at least one nonionic surfactant can be a mixture comprising an ethoxylated fatty ester, for instance PEG-40 stearate, and a sorbitan ester, such as sorbitan tristearate. The weight ratio of the fatty ester ethoxylated with 8 to 100 EO (for instance, from 10 to 80 EO) to the sorbitan ester of the at least one nonionic surfactant can range from 0.02 to 100, such as from 0.04 to 80.

The fatty ester ethoxylated with 8 to 100 EO can be present in an amount ranging from 0.01% to 10% by weight, for example, from 0.1% to 5% by weight, such as from 0.5% to 3% by weight, relative to the total weight of the composition.

The fatty acid ester of sorbitan can be present in an amount ranging from 0.1% to 10% by weight, for instance from 0.5% to 5% by weight, relative to the total weight of the composition.

The surfactant system can comprise at least one ionic surfactant with a dynamic interface tension of less than or equal to 7 millinewtons/meter. The dynamic interface tension of a surfactant is determined according to a protocol described herein.

Among ionic surfactants with a dynamic interface tension of less than 7 millinewtons/meter, non-limiting mention may be made of alkali metal salts of cetyl phosphate and alkali metal salts of palmitoyl sarcosinate, and mixtures thereof. The alkali metal salts can be, for example, the sodium salts or the potassium salts. In one embodiment of the present disclosure, the at least one ionic surfactant is chosen from potassium cetyl phosphate and sodium palmitoyl sarcosinate.

The weight ratio of the at least one ionic surfactant with a dynamic interface tension of less than or equal to 7 millinewtons/meter to the at least one nonionic surfactant within the surfactant system ranges from 0.02 to 75, for example, from 0.02 to 10.

The at least one ionic surfactant can be present in an amount ranging from 0.05% to 10% by weight, such as from 0.2% to 5%, and for instance, from 0.5% to 3% by weight, relative to the total weight of the composition.

The composition according to the present disclosure comprises an oily phase. The weight ratio of the surfactant system to the oily phase can range from 6×10⁻³ to 60, such as from 0.4 to 19.

The oily phase can be present in an amount ranging from 0.5% to 40% by weight, such as from 5% to 30% by weight, relative to the total weight of the composition.

In one embodiment of the present disclosure, the oily phase is present in an amount of at least 20% by weight, for example, from 20% to 40% by weight, relative to the total weight of the composition.

The oily phase of the emulsion according to the present disclosure comprises at least one oil chosen from oils of animal origin, oils of plant origin, mineral oils, synthetic oils, silicone oils, hydrocarbons such as aliphatic hydrocarbons, and mixtures thereof. These oils may be polar or non-polar, and volatile or non-volatile.

Among the polar oils that may be used as disclosed herein, non-limiting mention may be made of hydrocarbon-based oils comprising ester, ether, acid or alcohol functional groups or mixtures thereof, such as, for example:
hydrocarbon-based plant oils with a high content of triglyceride, comprising fatty acid esters of glycerol, the fatty acids of which may have varied chain lengths, wherein the chains are possibly linear or branched, and saturated or unsaturated; these oils can be; for example, wheatgerm oil, maize oil, sunflower oil, shea butter oil, castor oil, sweet almond oil, macadamia oil, apricot oil, soybean oil, rapeseed oil, cottonseed oil, alfalfa oil, poppy oil, pumpkin oil, sesame oil, marrow oil, avocado oil, hazelnut oil, grapeseed oil, blackcurrant seed oil, evening primrose oil, millet oil, barley oil, quinoa oil, olive oil, rye oil, safflower oil, candlenut oil, passion flower oil, or musk rose oil, or alternatively caprylic/capric acid triglycerides, for instance those sold by the company Stéarineries Dubois or those sold under the name Miglyol 810, 812 or 818 by the company Dynamit Nobel;
synthetic oils of formula R¹COOR², wherein R¹ is chosen from linear and branched higher fatty acid residues comprising from 7 to 19 carbon atoms and R² is chosen from branched hydrocarbon-based chains comprising from 3 to 20 carbon atoms, such as, for example, purcellin oil (cetostearyl octanoate), isononyl isononanoate or alkyl (C₁₂ to C₁₅) benzoates;
synthetic esters and synthetic ethers, for instance isopropyl myristate, 2-ethylhexyl palmitate and octanoates, decanoates or ricinoleates of alcohols or of polyalcohols; and
hydroxylated esters, for instance isostearyl lactate, diisostearyl malate and pentaerythritol esters.

Among the apolar oils that may be used as disclosed herein, non-limiting mention may be made of:
volatile or non-volatile, linear or cyclic silicone oils that are liquid at room temperature, such as polydimethylsiloxanes (PDMSs) comprising alkyl, alkoxy or phenyl groups, pendent and/or at the end of a silicone chain and comprising from 2 to 24 carbon atoms; phenylsilicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; and
linear or branched hydrocarbons or fluorohydrocarbons or fluorocarbons of synthetic or mineral origin, for instance volatile oils, such as liquid paraffins (for example isoparaffins), and aliphatic hydrocarbons (for example isododecane), or non-volatile oils and derivatives thereof; petroleum jelly, polydecenes, hydrogenated polyisobutene such as parleam oil, and squalane, and mixtures thereof.

The oily phase may also comprise fatty substances other than the oils mentioned above, such as one or more fatty alcohols, for instance stearyl alcohol, cetyl alcohol or behenyl alcohol, fatty acids such as stearic acid, palmitic acid and behenic acid, waxes such as glyceryl mono-, di- and tristearates or palmitates, gums, and mixtures thereof. When it is present, this other fatty substance, such as cetyl alcohol, may be present, for example, in an amount ranging from 2% to 10% by weight, for example from 2% to 5% by weight, relative to the total weight of the emulsion.

The composition according to the present disclosure comprises solid particles dispersed in the aqueous phase of the emulsion. The solid particles can be chosen from pigments, fillers, and mixtures thereof. In one embodiment of the present disclosure, the solid particles comprise at least pigments.

The solid particles present in the composition as disclosed herein can be dispersed in the form of particles with a number-average size of less than or equal to 10 μm (such as ranging from 0.5 μm to 10 μm), for example, less than or equal to 5 μm (such as ranging from 0.5 μm to 5 μm) and for instance, less than or equal to 2 μm (such as ranging from 0.5 μm to 2 μm).

The size of the solid particles dispersed in the composition may be measured with a granulometer of FPIA 2100 type sold by the company Malvern. The sample of the composition used to take the measurement is diluted in water such that the granulometer detects not more than 30,000 particles.

The solid particles dispersed in the composition can be present in an amount ranging from 1% to 25% by weight, for example, ranging from 2% to 15% by weight, such as ranging from 3% to 10% by weight, for instance ranging from 5% to 10% by weight, relative to the total weight of the composition.

The pigments may be chosen from mineral pigments, organic pigments and composite pigments (i.e., pigments based on mineral and/or organic materials).

The mineral pigments can be chosen from metal oxide pigments, mica coated with titanium dioxide, mica coated with bismuth oxychloride, titanium mica coated with iron oxide, titanium mica coated with ferric blue, titanium mica coated with chromium oxide, and mixtures thereof.

The metal oxide pigments can be chosen from, for example, iron oxides, titanium dioxide, zinc oxides, zirconium oxides, cerium oxides, and mixtures thereof. In one embodiment of the present disclosure, the mineral pigments are metal oxide pigments. In another embodiment of the present disclosure, for example, the mineral pigments are chosen from iron oxides, zinc oxides, zirconium oxides, cerium oxides, mica coated with titanium dioxide, mica coated with bismuth oxychloride, titanium mica coated with iron oxide, titanium mica coated with ferric blue, titanium mica coated with chromium oxide, organic pigments, and mixtures thereof.

The composition can also comprise titanium dioxide.

Among the organic particles intended to be coated, non-limiting examples include:
cochineal carmine;
organic pigments of azo dyes, anthraquinone dyes, indigoid dyes, xanthene dyes, pyrene dyes, quinoline dyes, triphenylmethane dyes or fluorane dyes;
organic lakes or insoluble salts of sodium, potassium, calcium, barium, aluminium, zirconium, strontium or titanium, or of acidic dyes such as azo dyes, anthraquinone dyes, indigoid dyes, xanthene dyes, pyrene dyes, quinoline dyes, triphenylmethane dyes or fluorane dyes. These dyes can comprise at least one carboxylic or sulfonic acid group; and
melanin pigments.

The organic lakes suitable for use as disclosed herein may also be supported by any compatible support such as a mineral support, for instance particles of alumina, of clay, of zirconia, of metal oxides, such as of zinc oxide or titanium oxide, of talc, of calcium carbonate or of barium sulfate. For example, the mineral support may be chosen from alumina, titanium oxide and barium sulfate. The organic lakes suitable for use as disclosed herein may also be supported by a support such as rosin or aluminium benzoate.

Among the organic pigments that may be used as disclosed herein, non-limiting mention may be made of D&C Blue No 4, D&C Brown No 1, D&C Green No 5, D&C Green No 6, D&C Orange No 4, D&C Orange No 5, D&C Orange No 10, D&C Orange No 11, D&C Red No 6, D&C Red No 7, D&C Red No 17, D&C Red No 21, D&C Red No 22, D&C Red No 27, D&C Red No 28, D&C Red No 30, D&C Red No 31, D&C Red No 33, D&C Red No 34, D&C Red No 36, D&C Violet No 2, D&C Yellow No 7, D&C Yellow No 8, D&C Yellow No 10, D&C Yellow No 11, FD&C Blue No 1, FD&C Green No 3, FD&C Red No 40, FD&C Yellow No 5 and FD&C Yellow No 6.

Among the organic lakes that may be used as disclosed herein, non-limiting mention may be made of, for example, those known under the following names:
D&C Red No 2 Aluminium lake,
D&C Red No 3 Aluminium lake,
D&C Red No 4 Aluminium lake,
D&C Red No 6 Aluminium lake,
D&C Red No 6 Barium lake,
D&C Red No 6 Barium/Strontium lake,
D&C Red No 6 Strontium lake,
D&C Red No 6 Potassium lake,
D&C Red No 7 Aluminium lake,
D&C Red No 7 Barium lake,
D&C Red No 7 Calcium lake,
D&C Red No 7 Calcium/Strontium lake,
D&C Red No 7 Zirconium lake,
D&C Red No 8 Sodium lake,
D&C Red No 9 Aluminium lake,
D&C Red No 9 Barium lake,
D&C Red No 9 Barium/Strontium lake,
D&C Red No 9 Zirconium lake,
D&C Red No 10 Sodium lake,
D&C Red No 19 Aluminium lake,
D&C Red No 19 Barium lake,
D&C Red No 19 Zirconium lake,
D&C Red No 21 Aluminium lake,
D&C Red No 21 Zirconium lake,
D&C Red No 22 Aluminium lake,
D&C Red No 27 Aluminium lake,
D&C Red No 27 Aluminium/Titanium/Zirconium lake,
D&C Red No 27 Barium lake,
D&C Red No 27 Calcium lake,
D&C Red No 27 Zirconium lake,
D&C Red No 28 Aluminium lake,
D&C Red No 30 lake,
D&C Red No 31 Calcium lake,
D&C Red No 33 Aluminium lake,
D&C Red No 34 Calcium lake,
D&C Red No 36 lake,
D&C Red No 40 Aluminium lake,
D&C Blue No 1 Aluminium lake,
D&C Green No 3 Aluminium lake,
D&C Orange No 4 Aluminium lake,
D&C Orange No 5 Aluminium lake,
D&C Orange No 5 Zirconium lake,
D&C Orange No 10 Aluminium lake,
D&C Orange No 17 Barium lake,
D&C Yellow No 5 Aluminium lake,
D&C Yellow No 5 Zirconium lake,
D&C Yellow No 6 Aluminium lake,
D&C Yellow No 7 Zirconium lake,
D&C Yellow No 10 Aluminium lake,
FD&C Blue No 1 Aluminium lake,
FD&C Red No 4 Aluminium lake,
FD&C Red No 40 Aluminium lake,
FD&C Yellow No 5 Aluminium lake, and
FD&C Yellow No 6 Aluminium lake.

The chemical compounds corresponding to each of the organic pigments mentioned above are mentioned in the publication International Cosmetic Ingredient Dictionary and Handbook, 1997 edition, pages 371 to 386 and 524 to 528, published by The Cosmetic, Toiletry and Fragrance Association, the content of which is incorporated into the present disclosure by reference.

Non-limiting examples of melanin pigments that may be used according to the present disclosure include, for example:
melanin pigments derived from natural or synthetic sources, which may be obtained: (A) by oxidation of at least one indole or indoline compound, or (B) by oxidative or enzymatic polymerization of melanin precursors, or (C) by extraction of melanin from substances containing it, or (D) by culturing microorganisms. Such melanin pigments are described for example, in European Patent No. EP-A-518 773, and PCT Publication Nos. WO-A-93/13744 and WO-A-93/13745.

The pigments may be present in the composition in an amount ranging from 0.5% to 25% by weight, for example, ranging from 1% to 12% by weight such as from 3% to 8% by weight, relative to the total weight of the composition.

The solid particles dispersed in the aqueous phase of the composition may also be fillers. The term “fillers” should be understood as meaning colorless or white, mineral or synthetic particles of any form, which are insoluble in the medium of the composition irrespective of the temperature at which the composition is manufactured and stored.

The fillers can be chosen for example, from mineral or organic fillers of any form, platelet, spherical or oblong, irrespective of the crystallographic form (for example lamellar, cubic, hexagonal, orthorhombic, etc.). Non-limiting mention may be made of talc, mica, silica, kaolin, polyamide (Nylon®) powder, poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie), or of acrylic acid copolymers, silicone resin microbeads (for example Tospearls® from Toshiba), precipitated calcium carbonate, magnesium carbonate, magnesium hydrocarbonate, hydroxyapatite, hollow silica microspheres, ceramic microcapsules, metal soaps derived from organic carboxylic acids comprising from 8 to 22 carbon atoms, such as from 12 to 18 carbon atoms, for example zinc stearate, magnesium stearate, lithium stearate, zinc laurate or magnesium myristate.

The fillers may be present in the composition in an amount ranging from 1% to 15% by weight, for example, ranging from 1% to 12% by weight, such as ranging from 2% to 8% by weight, relative to the total weight of the composition.

The solid particles, such as the pigments and fillers, can be, for example, dispersed in the aqueous phase of the composition in the presence of at least one ionic surfactant (also known as a dispersing surfactant) with a dynamic interface tension of less than 7 millinewtons/meter.

The at least one dispersing ionic surfactant can be, for example, an anionic surfactant.

Among the dispersing ionic surfactants with a dynamic interface tension of less than 7 millinewtons/meter that may be used as disclosed herein, non-limiting mention may be made of alkali metal salts of cetyl phosphate and alkali metal salts of palmitoyl sarcosinate, and mixtures thereof. The alkali metal salts are, for example, the sodium salts or the potassium salts. In one embodiment of the present disclosure, the at least one dispersing surfactant is chosen from potassium cetyl phosphate and sodium palmitoyl sarcosinate, and mixtures thereof.

For example, the solid particles can be predispersed with the at least one dispersing ionic surfactant with a dynamic interface tension of less than 7 millinewtons/meter in a fraction of the aqueous phase of the composition to form a pigmentary paste, this paste being added to the emulsion also prepared beforehand, in accordance with the process for preparing the composition described hereinbelow.

The at least one ionic surfactant with a dynamic interface tension of less than 7 millinewtons/meter that makes it possible to disperse the solid particles is present in the composition in an amount that is effective to be able to obtain a stable pigmented composition. In practice, this amount can range from 0.1% to 3% by weight, such as ranging from 0.3% to 2% by weight, for instance ranging from 0.5% to 1.8% by weight, relative to the total weight of the composition.

For example, the composition according to the present disclosure can comprise at least one nonionic aqueous thickener. This thickener can help promote good stability of the composition.

The at least one nonionic aqueous thickener can be chosen from:
cellulose polymers such as hydroxyethylcellulose, methylcellulose and hydroxypropylcellulose. Among these, non-limiting mention may be made of, for instance, the gums sold under the name “Cellosize QP 4400 H” by the company Amerchol;
nonionic guar gums comprising C₁-C₆ hydroxyalkyl groups. Non-limiting examples that may be mentioned include hydroxymethyl, hydroxyethyl, hydroxypropyl and hydroxybutyl groups. Such guar gums are sold for instance, under the trade names Jaguar HP8, Jaguar HP60, Jaguar HP120 and Jaguar HP105 by the company Meyhall, or under the name Galactosol 40H4FD2 by the company Aqualon;
carrageenans;
carob gum, scleroglucan gum, gellan gum, rhamsan gum and karaya gum;
vinyl polymers, for instance polyvinylpyrrolidones, copolymers of methyl vinyl ether and of malic anhydride, copolymers of vinylpyrrolidone and of vinyl acetate; copolymers of vinylpyrrolidone and of caprolactam; polyvinyl alcohol;
associative polymers such as associative polyurethanes;
and mixtures thereof.

For example, in one embodiment of the present disclosure, the thickener is an associative polyurethane.

Associative polyurethanes are nonionic block copolymers comprising in the chain both hydrophilic blocks usually of polyoxyethylenated nature and hydrophobic blocks that may be aliphatic sequences alone and/or cycloaliphatic and/or aromatic sequences.

For instance, these polymers can comprise at least two hydrocarbon-based lipophilic chains comprising from C₆ to C₃₀ carbon atoms, separated by a hydrophilic block, the hydrocarbon-based chains possibly being pendent chains or chains at the end of a hydrophilic block. For example, it is possible for at least one pendent chain to be envisaged. In addition, the polymer may comprise a hydrocarbon-based chain at one end or at the two ends of a hydrophilic block.

The polymers can be block copolymers in triblock or multiblock form. The hydrophobic blocks may thus be at each end of the chain (for example: triblock copolymer comprising a hydrophilic central block) or distributed both at the ends and within the chain (for example multiblock copolymer). The polymers may also be graft polymers or starburst polymers.

For example, the polymers can be triblock copolymers whose hydrophilic block is a polyoxyethylene chain comprising from 50 to 1000 oxyethylene groups. In general, the associative pqlyurethanes comprise a urethane bond between the hydrophilic blocks, whence arises the name.

By way of example, among the associative polymers that may be used as disclosed herein, non-limiting mention may be made of the polymer C₁₆—OE₁₂₀-C₁₆ sold by the company Hüils (under the name Serad FX1100, which is a molecule comprising a urethane functional group and having a weight-average molecular weight of 1300), OE being an oxyethylene unit. An associative polymer that may also be used is Rheolate 205 comprising a urea function, sold by the company Rheox, or Rheolate 208 or 204. These associative polyurethanes are sold in pure form. The product DW 1206B from Rohm & Haas comprising a C₂₀ alkyl chain with a urethane bond, sold at a solids content of 20% in water, may also be used.

It is also possible to use solutions or dispersions of these polymers, for instance, in water or in aqueous-alcoholic medium. Non-limiting examples of such polymers that may be mentioned include Serad FX1010, Serad FX1035 and Serad 1070 sold by the company Hüls, and Rheolate 255, Rheolate 278 and Rheolate 244 sold by the company Rheox. It is also possible to use the product DW 1206F and DW 1206J, or alternatively Borchigel LW 44 from the company Borchers.

The polymers described in the article by G. Fonnum, J. Bakke and Fk. Hansen—Colloid Polym. Sci 271, 380.389 (1993), may also be used in the composition of the present disclosure.

The nonionic aqueous thickener may be present in the composition according to the present disclosure in an amount ranging from 0.01% to 5% by weight, for instance ranging from 0.01% to 3% by weight, such as from 0.01% to 1.5% by weight, relative to the total weight of the composition.

The composition according to the present disclosure generally has the consistency of a gel or a cream. The viscosity of the composition can range from 1 to 70 poises (0.1 to 7 Pa.s), such as from 5 to 50 poises (0.5 to 5 Pa.s), the viscosity being measured at 25° C. with a Rheomat 180 viscometer (spindle 3).

The composition according to the present disclosure can also comprise other common cosmetic ingredients, which may be chosen from, for example, antioxidants, fragrances, preserving agents, neutralizers, waxes, sunscreens, vitamins, moisturizers, self-tanning compounds and antiwrinkle active agents.

The composition according to the present disclosure can comprise at least one water-soluble or liposoluble active agent with cosmetic or dermatological activity.

When present in the composition, the at least one liposoluble active agent is in the oily globules of the emulsion, while the at least one water-soluble active agent is in the aqueous phase of the emulsion. Non-limiting examples of active agents that may be mentioned include vitamins, such as vitamin E, vitamin C, vitamin A, vitamin PP and derivatives thereof, for instance esters thereof, provitamins such as panthenol, humectants and sunscreens.

The composition according to the present disclosure may be in a form chosen from a foundation, a lip makeup product (lipstick or lip gloss), an eyeshadow, a makeup rouge, a concealer product, a body makeup product, an eyeliner, and a mascara.

Still another aspect of the present disclosure is a process for preparing a makeup composition as disclosed herein, comprising:
preparing: an emulsion, such as a nanoemulsion, by mixing the aqueous phase and the oily phase with vigorous stirring, at a temperature ranging from 60 to 95° C., and then homogenizing the mixture at a pressure ranging from 4×10⁷ Pa to 18×10⁷ Pa, for instance ranging from 6×10⁷ Pa to 18×10⁷ Pa (high-pressure homogenization);
separately preparing a pigmentary paste, by dispersing solid particles, such as pigments or fillers, in an aqueous phase in the presence of ionic surfactant with a dynamic interface tension of less than 7 millinewtons/meter; and
introducing the pigmentary paste into the emulsion with moderate stirring so as to conserve the structure of the emulsion of the composition.

The pigmentary paste comprises, for example, from 15% to 75% by weight, for instance from 30% to 65% by weight of pigments, and from 0.5% to 10% by weight, for instance from 3% to 7% by weight of the ionic surfactant, relative to the total weight of the pigmentary paste.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The following examples are intended to illustrate the invention in a non-limiting manner. In the examples, unless otherwise indicated, the percentages and parts are expressed on a weight basis.

Method for Measuring the Dynamic Interface Tension of a Surfactant:

The dynamic interface tension of the surfactant was measured using a tensiometer, for example the tensiometer sold under the name Drop Volume Tensiometer model DVT-10 by the company Kruss.

A sample of a water and surfactant mixture comprising 0.5% by weight of surfactant, relative to the total weight of the mixture, was placed in a tube, at the bottom of which was fitted a capillary tube, connected to a precision syringe comprising an oil (the chosen oil generally corresponds to the majority oil present in the composition according to the present disclosure; in the composition examples mentioned below, the majority oil is hydrogenated polyisobutene, or parleam oil). The filled tube was placed in a water bath at 70° C. such that the water and surfactant mixture was not opaque. An Archimedean screw activated the syringe so as to push the oil into the capillary at a constant flow rate of 1 ml/h. At the orifice of the capillary (orifice of diameter d), a drop of oil formed, and then became detached and rose to the surface of the oil and surfactant mixture.

A luminescent electronic diode and a photodiode, positioned on the tube, detected the drops of oil that became detached from the capillary.

The time elapsed between the formation of two consecutive drops was measured using a chronometer. Since the flow rate is constant, the time was then converted to determine the volume of each drop, this volume being directly dependent on the dynamic interface tension σᵢ. The said tension was then calculated using the following equation:
σᵢ=V(ρ_{H}-ρ_{L})g/πd
wherein:
V is the volume of the drop, expressed in cm³
ρ_{H} is the density of the water and surfactant mixture, expressed in g/cm³
ρ_{L} is the density of the oil, expressed in g/cm³
g is the acceleration due to gravity, the value of which is 9.8 m/s²
d is the diameter of the orifice of the capillary, expressed in meters
ρ_{H} and ρ_{L} were measured conventionally using a densimeter
σᵢ was expressed in mN/m.

Ten time intervals between two drops were measured and the result obtained was a mean of these ten measurements.

This process applies for surfactants that are soluble in water at a temperature of 70° C., i.e., the water and surfactant mixture is not opaque at 70° C.

When the surfactant is insoluble in water, the process is inverted, i.e., the surfactant is first mixed with the oil and the syringe is filled with water.

### EXAMPLES OF COMPOSITIONS

The nanoemulsions of Examples 1 and 2 below were obtained by forming a coarse pre-emulsion using a rotor-stator, and then by adding the aqueous phase A to the oily phase B, at 80° C. The premix was then homogenized five times in a high-pressure homogenizer (Soavi type OBL 20) with a first-stage pressure of 4×10⁷ Pa (1100 bar) and a second-stage pressure of 7×10⁶ Pa (120 bar), with cooling to 70° C. at the outlet.

### Example 1

A nanoemulsion having the composition below was prepared:

| | | |
|---|---|---|
| A | Water | 60.05% |
| | Methyl paraben | 0.2% |
| B | Sorbitan tristearate (1) | 1% |
| | Cetyl alcohol | 4.7% |
| | Glyceryl stearate (2) | 3.9% |
| | Polyethylene glycol stearate (40 EO) | 2.22% |
| | Hydrogenated polyisobutene (Parleam | 13.5% |
| | oil) | |
| | Isononyl isononanoate | 13.5% |
| | Potassium cetyl phosphate | 0.83% |
| | Propyl paraben | 0.1% |

(1) Span 65V from Uniqema
(2) Glyceryl stearate codex from Stéarinerie Dubois

### Example 2

A nanoemulsion having the composition below was prepared:

| | | |
|---|---|---|
| A | Water | 60.56% |
| | Methyl paraben | 0.2% |
| B | Sorbitan tristearate (1) | 1.28% |
| | Cetyl alcohol | 5.72% |
| | Glyceryl stearate (2) | 4.71% |
| | Polyethylene glycol stearate (40 EO) | 2.86% |
| | Hydrogenated polyisobutene (Parleam | 23.6% |
| | oil) | |
| | Potassium cetyl phosphate | 1.07% |

(1) Span 65V from Uniqema
(2) Glyceryl stearate codex from Stéarinerie Dubois

### Example 3

A nanoemulsion having the composition below was prepared:

| | | |
|---|---|---|
| A | Water | 60.75% |
| | Methyl paraben | 0.2% |
| B | Sorbitan tristearate (1) | 1% |
| | Cetyl alcohol | 4% |
| | Glyceryl stearate (2) | 3.9% |
| | Polyethylene glycol stearate (40 EO) | 2.22% |
| | Hydrogenated polyisobutene (Parleam | 10% |
| | oil) | |
| | Diisostearyl malate | 10% |
| | Potassium cetyl phosphate | 0.83% |
| | Propyl paraben | 0.1% |
| | Dimethicone (3) | 7% |

(1) Span 65V from Uniqema
(2) Glyceryl stearate codex from Stéarinerie Dubois
(3) Dow Corning DC 200 —5 cst from Dow Corning

The nanoemulsions of Examples 1 to 3 according to the present disclosure had the consistency of a gel and were stable during and after two months of accelerated ageing at 4° C., at room temperature, and at 45° C.

### Example 4

A pigmentary paste having the composition below was prepared:

| | |
|---|---|
| Titanium dioxide | 20% |
| Yellow iron oxides | 4.7% |
| Brown iron oxides | 4% |
| Black iron oxides | 1% |
| Sodium palmitoyl sarcosinate | 2.5% |
| Water | 67.8% |

The ingredients were all mixed together at room temperature and the mixture was then ground in a ball mill for 30 minutes at 25° C. at a speed of 1200 rpm. A stable homogeneous pigmentary paste was thus obtained.

A foundation having the composition below was prepared:
Nanoemulsion of Example 1 90%
Pigmentary paste according to a) 10%

The pigmentary paste was dispersed in the nanoemulsion with stirring using a straight paddle at 400 rpm.

A fluid foundation was obtained, with a viscosity of 6.5 poises (0.65 Pa.s), measured at 25° C. using a Rheomat 180 viscometer equipped with a No 3 spindle; the measurement was performed after spinning the spindle for 10 minutes.

This foundation was stable during and after storage for two months at 45° C., and had a light texture, both to touch and when applied to the skin. After application, the foundation was comfortable, and moisturized and softened the skin.

### Example 5

A pigmentary paste having the composition below was prepared:

| | |
|---|---|
| Titanium dioxide | 22.46% |
| Yellow iron oxides | 5.24% |
| Brown iron oxides | 4.5% |
| Black iron oxides | 1.14% |
| Sodium palmitoyl sarcosinate | 2.5% |
| Water | 64.16% |

The ingredients were all mixed together at room temperature and the mixture was then ground in a ball mill for 30 minutes at 25° C. at a speed of 1200 rpm. A stable homogeneous pigmentary paste was thus obtained.

A foundation having the composition below was prepared:
Nanoemulsion of Example 2 70%
Pigmentary paste according to Example 5 30%

The pigmentary paste was dispersed in the nanoemulsion with stirring using a straight paddle at 400 rpm.

A fluid foundation was obtained, with a viscosity of 8 poises (0.8 Pa.s), measured at 25° C. using a Rheomat 180 viscometer equipped with a No 3 spindle; the measurement was performed after spinning the spindle for 10 minutes.

This foundation was stable during and after storage for two months at 45° C., and had a light texture, both to touch and when applied to the skin. After application, the foundation was comfortable, and moisturized and softened the skin.

### Example 6

A pigmentary paste having the composition below was prepared:

| | |
|---|---|
| Titanium dioxide | 22.46% |
| Yellow iron oxides | 5.24% |
| Brown iron oxides | 4.5% |
| Black iron oxides | 1.14% |
| Sodium palmitoyl sarcosinate | 2.5% |
| Water | 64.16% |

The ingredients were all mixed together at room temperature and the mixture was then ground in a ball mill for 30 minutes at 25° C. at a speed of 1200 rpm. A stable homogeneous pigmentary paste was thus obtained.

A foundation having the composition below was prepared:

| | |
|---|---|
| Nanoemulsion of Example 1 | 86.2% |
| Pigmentary paste according to Example 6 | 13.75% |
| Associative polyurethane (Ser-Ad FX 1100 | 0.5% |
| from the company Servo Delden) | |

The pigmentary paste was dispersed in the nanoemulsion with stirring using a straight paddle at 400 rpm, and the polyurethane was then added.

A fluid foundation was obtained, with a viscosity of 9 poises (0.9 Pa.s), measured at 25° C. using a Rheomat 180 viscometer equipped with a No 3 spindle; the measurement was performed after spinning the spindle for 10 minutes.

This foundation was stable during and after storage for two months at 45° C., and had a light texture, both to touch and when applied to the skin. After application, the foundation was comfortable, and moisturized and softened the skin.

### Example 7

A pigmentary paste having the composition below was prepared:

| | |
|---|---|
| Titanium dioxide | 6.60% |
| Brown and yellow iron oxides | 12.73% |
| Black iron oxides | 5.44% |
| Calcium salt of red lithol B | 12.73% |
| Sodium palmitoyl sarcosinate | 5.79% |
| Water | 56.71% |

The ingredients were all mixed together at room temperature and the mixture was then ground in a ball mill for 30 minutes at 25° C. at a speed of 1200 rpm. A stable homogeneous pigmentary paste was thus obtained.

A lipstick having the composition below was prepared:

| | |
|---|---|
| Nanoemulsion of Example 3 | 79.5% |
| Associative polyurethane (Ser-Ad FX 1100 | 0.5% |
| from the company Servo Delden) | |
| Water | 5% |
| Pigmentary paste according to Example 7 | 10% |
| Glycerol | 5% |

The pigmentary paste and the glycerol were dispersed in the nanoemulsion with stirring using a straight paddle at 400 rpm, and the mixture of water and polyurethane was then added.

A fluid lipstick was obtained, with a viscosity of 30 poises (3 Pa.s), measured at 25° C. using a Rheomat 180 viscometer equipped with a No 3 spindle; the measurement was performed after spinning the spindle for 10 minutes.

This lipstick was stable during and after storage for two months at 45° C., and had a light texture when applied to the lips.

1. A makeup composition comprising:
(i) an emulsion comprising an oily phase dispersed in an aqueous phase, and
(ii) solid particles dispersed in the aqueous phase of the emulsion such that the makeup composition is stable after storage for two months at a temperature less than or equal to 45° C.,
wherein the emulsion comprises oil globules with a number-average size of less than or equal to 500 nm and further comprises a surfactant system comprising:
at least one nonionic surfactant, and
at least one ionic surfactant with a dynamic interface tension of less than or equal to 7 millinewtons/meter.

2. The composition according to claim 1, wherein the at least one nonionic surfactant is chosen from ethoxylated fatty esters comprising 8 to 100 ethylene oxide units and fatty acid esters of sorbitan.

3. The composition according to claim 1, wherein the at least one nonionic surfactant is present in an amount ranging from 1% to 10% by weight, relative to the total weight of the composition.

4. The composition according to claim 1, wherein the at least one ionic surfactant of the surfactant system is chosen from alkali metal salts of cetyl phosphate and alkali metal salts of palmitoyl sarcosinate.

5. The composition according to claim 4, wherein the at least one ionic surfactant of the surfactant system is chosen from potassium cetyl phosphate and sodium palmitoyl sarcosinate.

6. The composition according to claim 1, wherein the weight ratio of the at least one ionic surfactant to the at least one nonionic surfactant ranges from 0.02 to 75.

7. The composition according to claim 1, wherein the at least one ionic surfactant is present in an amount ranging from 0.05% to 10% by weight, relative to the total weight of the composition.

8. The composition according to claim 1, wherein the emulsion comprises oil globules with a number-average size of less than or equal to 300 nm.

9. The composition according to claim 1, wherein the oily phase is present in an amount ranging from 0.5% to 40% by weight, relative to the total weight of the emulsion.

10. The composition according to claim 1, wherein the oily phase comprises at least one oil chosen from oils of animal or plant origin, mineral oils, synthetic oils, silicone oils and aliphatic hydrocarbons.

11. The composition according to claim 1, further comprising at least one other fatty substance chosen from fatty alcohols, fatty acids, waxes and gums.

12. The composition according to claim 11, wherein the at least one other fatty substance is present in an amount ranging from 2% to 10% by weight, relative to the total weight of the emulsion.

13. The composition according to claim 11, wherein the at least one fatty alcohol is chosen from stearyl alcohol, cetyl alcohol and behenyl alcohol, and wherein the at least one fatty acid is chosen from stearic acid, palmitic acid and behenic acid.

14. The composition according to claim 13, wherein the at least one fatty alcohol is cetyl alcohol.

15. The composition according to claim 11, wherein the at least one other fatty substance is chosen from glyceryl mono-, di- or tristearate and glyceryl mono-, di- or tripalmitate.

16. The composition according to claim 1, wherein the solid particles are chosen from pigments and fillers, and mixtures thereof.

17. The composition according to claim 1, wherein the dispersed solid particles are present in an amount ranging from 1% to 25% by weight, relative to the total weight of the composition.

18. The composition according to claim 1, further comprising at least one nonionic aqueous thickener.

19. The composition according to claim 1, further comprising at least one adjuvant chosen from antioxidants, fragrances, preserving agents, neutralizers, waxes, sunscreens, vitamins, moisturizers, self-tanning compounds and antiwrinkle active agents.

20. The composition according to claim 1, wherein it is in a form chosen from a foundation, a lip makeup product, an eyeshadow, a makeup rouge, a concealer product, a body makeup product, an eyeliner, and a mascara.

21. A makeup composition comprising:
(i) an emulsion comprising an oily phase dispersed in an aqueous phase, and
(ii) solid particles having a number-average size of less than or equal to 10 μm dispersed in the aqueous phase of the emulsion,
wherein the emulsion comprises oil globules with a number-average size of less than or equal to 500 nm and also comprises a surfactant system comprising:
at least one nonionic surfactant, and
at least one ionic surfactant with a dynamic interface tension of less than or equal to 7 millinewtons/meter.

22. The composition according to claim 21, wherein the solid particles have a number-average size ranging from 0.5 μm to 10 μm.

23. The composition according to claim 21, wherein the at least one nonionic surfactant is chosen from ethoxylated fatty esters comprising 8 to 100 ethylene oxide units and fatty acid esters of sorbitan.

24. The composition according to claim 21, wherein the at least one nonionic surfactant is present in an amount ranging from 1% to 10% by weight, relative to the total weight of the composition.

25. The composition according to claim 21, wherein the at least one ionic surfactant of the surfactant system is chosen from alkali metal salts of cetyl phosphate and alkali metal salts of palmitoyl sarcosinate.

26. The composition according to claim 25, wherein the at least one ionic surfactant of the surfactant system is chosen from potassium cetyl phosphate and sodium palmitoyl sarcosinate.

27. The composition according to claim 21, wherein the weight ratio of the at least one ionic surfactant to the at least one nonionic surfactant ranges from 0.02 to 75.

28. The composition according to claim 21, wherein the at least one ionic surfactant is present in an amount ranging from 0.05% to 10% by weight, relative to the total weight of the composition.

29. The composition according to claim 21, wherein the emulsion comprises oil globules with a number-average size of less than or equal to 300 nm.

30. The composition according to claim 21, wherein the oily phase is present in an amount ranging from 0.5% to 40% by weight, relative to the total weight of the emulsion.

31. The composition according to claim 21, wherein the oily phase comprises at least one oil chosen from oils of animal or plant origin, mineral oils, synthetic oils, silicone oils and aliphatic hydrocarbons.

32. The composition according to claim 21, further comprising at least one other fatty substance chosen from fatty alcohols, fatty acids, waxes and gums.

33. The composition according to claim 31, wherein the at least one other fatty substance is present in an amount ranging from 2% to 10% by weight, relative to the total weight of the emulsion.

34. The composition according to claim 32, wherein the at least one fatty alcohol is chosen from stearyl alcohol, cetyl alcohol and behenyl alcohol, and wherein the at least one fatty acid is chosen from stearic acid, palmitic acid and behenic acid.

35. The composition according to claim 34, wherein the at least one fatty alcohol is cetyl alcohol.

36. The composition according to claim 32, wherein the at least one other fatty substance is chosen from glyceryl mono-, di- or tristearate and glyceryl mono-, di- or tripalmitate.

37. The composition according to claim 21, wherein the solid particles are chosen from pigments, fillers, and mixtures thereof.

38. The composition according to claim 21, wherein the dispersed solid particles are present in an amount ranging from 1% to 25% by weight, relative to the total weight of the composition.

39. The composition according to claim 21, further comprising at least one nonionic aqueous thickener.

40. The composition according to claim 21, further comprising at least one adjuvant chosen from antioxidants, fragrances, preserving agents, neutralizers, waxes, sunscreens, vitamins, moisturizers, self-tanning compounds and antiwrinkle active agents.

41. The composition according to claim 21, wherein it is in a form chosen from a foundation, a lip makeup product, an eyeshadow, a makeup rouge, a concealer product, a body makeup product, an eyeliner, and a mascara.

42. A makeup composition comprising:
(i) an emulsion comprising an oily phase dispersed in an aqueous phase, and
(ii) solid particles dispersed in the aqueous phase of the emulsion such that the makeup composition is stable after storage for two months at a temperature less than or equal to 45° C.,
wherein the emulsion comprises a surfactant system comprising:
at least one nonionic surfactant, and
at least one ionic surfactant with a dynamic interface tension of less than or equal to 7 millinewtons/meter, and
wherein the solid particles comprise at least one pigment chosen from iron oxides, zinc oxides, zirconium oxides, cerium oxides, mica coated with titanium dioxide, mica coated with bismuth oxychloride, titanium mica coated with iron oxide, titanium mica coated with ferric blue, titanium mica coated with chromium oxide, and organic pigments.

43. The composition according to claim 42, wherein the at least one nonionic surfactant is chosen from ethoxylated fatty esters comprising 8 to 100 ethylene oxide units and fatty acid esters of sorbitan.

44. The composition according to claim 42, wherein the at least one nonionic surfactant is present in an amount ranging from 1% to 10% by weight, relative to the total weight of the composition.

45. The composition according to claim 42, wherein the at least one ionic surfactant of the surfactant system is chosen from alkali metal salts of cetyl phosphate and alkali metal salts of palmitoyl sarcosinate.

46. The composition according to claim 45, wherein the at least one ionic surfactant of the surfactant system is chosen from potassium cetyl phosphate and sodium palmitoyl sarcosinate.

47. The composition according to claim 42, wherein the weight ratio of the at least one ionic surfactant to the at least one nonionic surfactant ranges from 0.02 to 75.

48. The composition according to claim 42, wherein the at least one ionic surfactant is present in an amount ranging from 0.05% to 10% by weight, relative to the total weight of the composition.

49. The composition according to claim 42, wherein the emulsion comprises oil globules with a number-average size of less than or equal to 500 nm.

50. The composition according to claim 42, wherein the oily phase is present in an amount ranging from 0.5% to 40% by weight, relative to the total weight of the emulsion.

51. The composition according to claim 42, wherein the oily phase comprises at least one oil chosen from oils of animal or plant origin, mineral oils, synthetic oils, silicone oils and aliphatic hydrocarbons.

52. The composition according to claim 42, further comprising at least one other fatty substance chosen from fatty alcohols, fatty acids, waxes and gums.

53. The composition according to claim 52, wherein the at least one other fatty substance is present in an amount ranging from 2% to 10% by weight, relative to the total weight of the emulsion.

54. The composition according to claim 52, wherein the at least one fatty alcohol is chosen from stearyl alcohol, cetyl alcohol and behenyl alcohol, and wherein the at least one fatty acid is chosen from stearic acid, palmitic acid and behenic acid.

55. The composition according to claim 54, wherein the at least one fatty alcohol is cetyl alcohol.

56. The composition according to claim 52, wherein the at least one other fatty substance is chosen from glyceryl mono-, di- or tristearate and glyceryl mono-, di- or tripalmitate.

57. The composition according to claim 42, wherein the dispersed solid particles are present in an amount ranging from 1% to 25% by weight, relative to the total weight of the composition.

58. The composition according to claim 42, further comprising at least one nonionic aqueous thickener.

59. The composition according to claim 42, further comprising at least one adjuvant chosen from antioxidants, fragrances, preserving agents, neutralizers, waxes, sunscreens, vitamins, moisturizers, self-tanning compounds and antiwrinkle active agents.

60. The composition according to claim 42, wherein it is in a form chosen from a foundation, a lip makeup product, an eyeshadow, a makeup rouge, a concealer product, a body makeup product, an eyeliner, and a mascara.

61. A makeup composition comprising:
(i) an emulsion comprising an oily phase dispersed in an aqueous phase, and
(ii) solid particles having a number-average size of less than or equal to 10 μm dispersed in the aqueous phase of the emulsion,
wherein the emulsion comprises a surfactant system comprising:
at least one nonionic surfactant, and
at least one ionic surfactant with a dynamic interface tension of less than or equal to 7 millinewtons/meter, and
wherein the solid particles comprise at least one pigment chosen from iron oxides, zinc oxides, zirconium oxides, cerium oxides, mica coated with titanium dioxide, mica coated with bismuth oxychloride, titanium mica coated with iron oxide, titanium mica coated with ferric blue, titanium mica coated with chromium oxide, and organic pigments.

62. The composition according to claim 61, wherein the solid particles have a number-average size ranging from 0.5 μm to 10 μm.

63. The composition according to claim 61, wherein the at least one nonionic surfactant is chosen from ethoxylated fatty esters comprising 8 to 100 ethylene oxide units and fatty acid esters of sorbitan.

64. The composition according to claim 61, wherein the at least one nonionic surfactant is present in an amount ranging from 1% to 10% by weight, relative to the total weight of the composition.

65. The composition according to claim 61, wherein the at least one ionic surfactant of the surfactant system is chosen from alkali metal salts of cetyl phosphate and alkali metal salts of palmitoyl sarcosinate.

66. The composition according to claim 65, wherein the at least one ionic surfactant of the surfactant system is chosen from potassium cetyl phosphate and sodium palmitoyl sarcosinate.

67. The composition according to claim 61, wherein the weight ratio of the at least one ionic surfactant to the at least one nonionic surfactant ranges from 0.02 to 75.

68. The composition according to claim 67, wherein the at least one ionic surfactant is present in an amount ranging from 0.05% to 10% by weight, relative to the total weight of the composition.

69. The composition according to claim 61, wherein the emulsion comprises oil globules with a number-average size of less than or equal to 500 nm.

70. The composition according to claim 61, wherein the oily phase is present in an amount ranging from 0.5% to 40% by weight, relative to the total weight of the emulsion.

71. The composition according to claim 61, wherein the oily phase comprises at least one oil chosen from oils of animal or plant origin, mineral oils, synthetic oils, silicone oils and aliphatic hydrocarbons.

72. The composition according to claim 61, further comprising at least one other fatty substance chosen from fatty alcohols, fatty acids, waxes and gums.

73. The composition according to claim 72, wherein the at least one other fatty substance is present in an amount ranging from 2% to 10% by weight, relative to the total weight of the emulsion.

74. The composition according to claim 72, wherein the at least one fatty alcohol is chosen from stearyl alcohol, cetyl alcohol and behenyl alcohol, and wherein the at least one fatty acid is chosen from stearic acid, palmitic acid and behenic acid.

75. The composition according to claim 74, wherein the at least one fatty alcohol is cetyl alcohol.

76. The composition according to claim 72, wherein the at least one other fatty substance is chosen from glyceryl mono-, di- or tristearate and glyceryl mono-, di- or tripalmitate.

77. The composition according to claim 61, wherein the dispersed solid particles are present in an amount ranging from 1% to 25% by weight, relative to the total weight of the composition.

78. The composition according to claim 61, further comprising at least one nonionic aqueous thickener.

79. The composition according to claim 61, further comprising at least one adjuvant chosen from antioxidants, fragrances, preserving agents, neutralizers, waxes, sunscreens, vitamins, moisturizers, self-tanning compounds and antiwrinkle active agents.

80. The composition according to claim 61, wherein it is in a form chosen from a foundation, a lip makeup product, an eyeshadow, a makeup rouge, a concealer product, a body makeup product, an eyeliner, and a mascara.

81. A makeup composition comprising:
(i) an emulsion comprising an oily phase dispersed in an aqueous phase, and
(ii) solid particles dispersed in the aqueous phase of the emulsion such that the makeup composition is stable after storage for two months at a temperature less than or equal to 45° C.,
wherein the emulsion comprises a surfactant system comprising:
at least one nonionic surfactant, and
at least one ionic surfactant with a dynamic interface tension of less than or equal to 7 millinewtons/meter, and
wherein the oily phase is present in amount of at least 20% by weight, relative to the total weight of the composition.

82. The composition according to claim 81, wherein the at least one nonionic surfactant is chosen from ethoxylated fatty esters comprising 8 to 100 ethylene oxide units and fatty acid esters of sorbitan.

83. The composition according to claim 81, wherein the at least one nonionic surfactant is present in an amount ranging from 1% to 10% by weight, relative to the total weight of the composition.

84. The composition according to claim 81, wherein the at least one ionic surfactant of the surfactant system is chosen from alkali metal salts of cetyl phosphate and alkali metal salts of palmitoyl sarcosinate.

85. The composition according to claim 84, wherein the at least one ionic surfactant of the surfactant system is chosen from potassium cetyl phosphate and sodium palmitoyl sarcosinate.

86. The composition according to claim 81, wherein the weight ratio of the at least one ionic surfactant to the at least one nonionic surfactant ranges from 0.02 to 75.

87. The composition according to claim 81, wherein the at least one ionic surfactant is present in an amount ranging from 0.05% to 10% by weight, relative to the total weight of the composition.

88. The composition according to claim 81, wherein the emulsion comprises oil globules with a number-average size of less than or equal to 500 nm.

89. The composition according to claim 81, wherein the oily phase is present in an amount ranging from 0.5% to 40% by weight, relative to the total weight of the emulsion.

90. The composition according to claim 81, wherein the oily phase comprises at least one oil chosen from oils of animal or plant origin, mineral oils, synthetic oils, silicone oils and aliphatic hydrocarbons.

91. The composition according to claim 81, further comprising at least one other fatty substance chosen from fatty alcohols, fatty acids, waxes and gums.

92. The composition according to claim 91, wherein the at least one other fatty substance is present in an amount ranging from 2% to 10% by weight, relative to the total weight of the emulsion.

93. The composition according to claim 91, wherein the at least one fatty alcohol is chosen from stearyl alcohol, cetyl alcohol and behenyl alcohol, and wherein the at least one fatty acid is chosen from stearic acid, palmitic acid and behenic acid.

94. The composition according to claim 93, wherein the at least one fatty alcohol is cetyl alcohol.

95. The composition according to claim 91, wherein the at least one other fatty substance is chosen from glyceryl mono-, di- or tristearate and glyceryl mono-, di- or tripalmitate.

96. The composition according to claim 81, wherein the solid particles are chosen from pigments, fillers, and mixtures thereof.

97. The composition according to claim 81, wherein the dispersed solid particles are present in an amount ranging from 1% to 25% by weight, relative to the total weight of the composition.

98. The composition according to claim 81, further comprising at least one nonionic aqueous thickener.

99. The composition according to claim 81, further comprising at least one adjuvant chosen from antioxidants, fragrances, preserving agents, neutralizers, waxes, sunscreens, vitamins, moisturizers, self-tanning compounds and antiwrinkle active agents.

100. The composition according to claim 81, wherein it is in a form chosen from a foundation, a lip makeup product, an eyeshadow, a makeup rouge, a concealer product, a body makeup product, an eyeliner, and a mascara.

101. A makeup composition comprising:
(i) an emulsion comprising an oily phase dispersed in an aqueous phase, and
(ii) solid particles having a number-average size of less than or equal to 10 μm dispersed in the aqueous phase of the emulsion,
wherein the emulsion comprises a surfactant system comprising:
at least one nonionic surfactant, and
at least one ionic surfactant with a dynamic interface tension of less than or equal to 7 millinewtons/meter, and
wherein the oily phase is present in an amount of at least 20% by weight, relative to the total weight of the composition.

102. The composition according to claim 101, wherein the solid particles have a number-average size ranging from 0.5 μm to 10 μm.

103. The composition according to claim 101, wherein the at least one nonionic surfactant is chosen from ethoxylated fatty esters comprising 8 to 100 ethylene oxide units and fatty acid esters of sorbitan.

104. The composition according to claim 101, wherein the at least one nonionic surfactant is present in an amount ranging from 1% to 10% by weight, relative to the total weight of the composition.

105. The composition according to claim 101, wherein the at least one ionic surfactant of the surfactant system is chosen from alkali metal salts of cetyl phosphate and alkali metal salts of palmitoyl sarcosinate.

106. The composition according to claim 105, wherein the at least one ionic surfactant of the surfactant system is chosen from potassium cetyl phosphate and sodium palmitoyl sarcosinate.

107. The composition according to claim 101, wherein the weight ratio of the at least one ionic surfactant to the at least one nonionic surfactant ranges from 0.02 to 75.

108. The composition according to claim 101, wherein the at least one ionic surfactant is present in an amount ranging from 0.05% to 10% by weight, relative to the total weight of the composition.

109. The composition according to claim 101, wherein the emulsion comprises oil globules with a number-average size of less than or equal to 500 nm.

110. The composition according to claim 101, wherein the oily phase is present in an amount ranging from 0.5% to 40% by weight, relative to the total weight of the emulsion.

111. The composition according to claim 101, wherein the oily phase comprises at least one oil chosen from oils of animal or plant origin, mineral oils, synthetic oils, silicone oils and aliphatic hydrocarbons.

112. The composition according to claim 101, further comprising at least one other fatty substance chosen from fatty alcohols, fatty acids, waxes and gums.

113. The composition according to claim 112, wherein the at least one other fatty substance is present in an amount ranging from 2% to 10% by weight, relative to the total weight of the emulsion.

114. The composition according to claim 112, wherein the at least one fatty alcohol is chosen from stearyl alcohol, cetyl alcohol and behenyl alcohol, and wherein the at least one fatty acid is chosen from stearic acid, palmitic acid and behenic acid.

115. The composition according to claim 114, wherein the at least one fatty alcohol is cetyl alcohol.

116. The composition according to claim 112, wherein the at least one other fatty substance is chosen from glyceryl mono-, di- or tristearate and glyceryl mono-, di- or tripalmitate.

117. The composition according to claim 101, wherein the solid particles are chosen from pigments, fillers, and mixtures thereof.

118. The composition according to claim 101, wherein the dispersed solid particles are present in an amount ranging from 1% to 25% by weight, relative to the total weight of the composition.

119. The composition according to claim 101, further comprising at least one nonionic aqueous thickener.

120. The composition according to claim 101, further comprising at least one adjuvant chosen from antioxidants, fragrances, preserving agents, neutralizers, waxes, sunscreens, vitamins, moisturizers, self-tanning compounds and antiwrinkle active agents.

121. The composition according to claim 101, wherein it is in a form chosen from a foundation, a lip makeup product, an eyeshadow, a makeup rouge, a concealer product, a body makeup product, an eyeliner, and a mascara.

122. A makeup composition comprising:
(i) an emulsion comprising an oily phase dispersed in an aqueous phase,
(ii) solid particles dispersed in the aqueous phase of the emulsion such that the makeup composition is stable after storage for two months at a temperature less than or equal to 45° C., and
(iii) a nonionic aqueous thickener chosen from associative polyurethanes,
wherein the emulsion comprises a surfactant system comprising:
at least one nonionic surfactant, and
at least one ionic surfactant with a dynamic interface tension of less than or equal to 7 millinewtons/meter.

123. The composition according to claim 122, wherein the at least one nonionic surfactant is chosen from ethoxylated fatty esters comprising 8 to 100 ethylene oxide units and fatty acid esters of sorbitan.

124. The composition according to claim 122, wherein the at least one nonionic surfactant is present in an amount ranging from 1% to 10% by weight, relative to the total weight of the composition.

125. The composition according to claim 122, wherein the at least one ionic surfactant of the surfactant system is chosen from alkali metal salts of cetyl phosphate and alkali metal salts of palmitoyl sarcosinate.

126. The composition according to claim 125, wherein the at least one ionic surfactant of the surfactant system is chosen from potassium cetyl phosphate and sodium palmitoyl sarcosinate.

127. The composition according to claim 122, wherein the weight ratio of the at least one ionic surfactant to the at least one nonionic surfactant ranges from 0.02 to 75.

128. The composition according to claim 122, wherein the at least one ionic surfactant is present in an amount ranging from 0.05% to 10% by weight, relative to the total weight of the composition.

129. The composition according to claim 122, wherein the emulsion comprises oil globules with a number-average size of less than or equal to 500 nm.

130. The composition according to claim 122, wherein the oily phase is present in an amount ranging from 0.5% to 40% by weight, relative to the total weight of the emulsion.

131. The composition according to claim 122, wherein the oily phase comprises at least one oil chosen from oils of animal or plant origin, mineral oils, synthetic oils, silicone oils and aliphatic hydrocarbons.

132. The composition according to claim 122, further comprising at least one other fatty substance chosen from fatty alcohols, fatty acids, waxes and gums.

133. The composition according to claim 132, wherein the at least one other fatty substance is present in an amount ranging from 2% to 10% by weight, relative to the total weight of the emulsion.

134. The composition according to claim 132, wherein the at least one fatty alcohol is chosen from stearyl alcohol, cetyl alcohol and behenyl alcohol, and wherein the at least one fatty acid is chosen from stearic acid, palmitic acid and behenic acid.

135. The composition according to claim 134, wherein the at least one fatty alcohol is cetyl alcohol.

136. The composition according to claim 132, wherein the at least one other fatty substance is chosen from glyceryl mono-, di- or tristearate and glyceryl mono-, di- or tripalmitate.

137. The composition according to claim 122, wherein the solid particles are chosen from pigments, fillers, and mixtures thereof.

138. The composition according to claim 122, wherein the dispersed solid particles are present in an amount ranging from 1% to 25% by weight, relative to the total weight of the composition.

139. The composition according to claim 122, further comprising at least one adjuvant chosen from antioxidants, fragrances, preserving agents, neutralizers, waxes, sunscreens, vitamins, moisturizers, self-tanning compounds and antiwrinkle active agents.

140. The composition according to claim 122, wherein it is in a form chosen from a foundation, a lip makeup product, an eyeshadow, a makeup rouge, a concealer product, a body makeup product, an eyeliner, and a mascara.

141. A makeup composition comprising:
(i) an emulsion comprising an oily phase dispersed in an aqueous phase,
(ii) solid particles having a number-average size of less than or equal to 10 μm dispersed in the aqueous phase of the emulsion, and
(iii) a nonionic aqueous thickener chosen from associative polyurethanes,
wherein the emulsion comprises a surfactant system comprising:
at least one nonionic surfactant, and
at least one ionic surfactant with a dynamic interface tension of less than or equal to 7 millinewtons/meter.

142. The composition according to claim 141, wherein the solid particles have a number-average size ranging from 0.5 μm to 10 μm.

143. The composition according to claim 141, wherein the at least one nonionic surfactant is chosen from ethoxylated fatty esters comprising 8 to 100 ethylene oxide units and fatty acid esters of sorbitan.

144. The composition according to claim 141, wherein the at least one nonionic surfactant is present in an amount ranging from 1% to 10% by weight, relative to the total weight of the composition.

145. The composition according to claim 141, wherein the at least one ionic surfactant of the surfactant system is chosen from alkali metal salts of cetyl phosphate and alkali metal salts of palmitoyl sarcosinate.

146. The composition according to claim 145, wherein the at least one ionic surfactant of the surfactant system is chosen from potassium cetyl phosphate and sodium palmitoyl sarcosinate.

147. The composition according to claim 141, wherein the weight ratio of the at least one ionic surfactant to the at least one nonionic surfactant ranges from 0.02 to 75.

148. The composition according to claim 141, wherein the at least one ionic surfactant is present in an amount ranging from 0.05% to 10% by weight, relative to the total weight of the composition.

149. The composition according to claim 141, wherein the emulsion comprises oil globules with a number-average size of less than or equal to 500 nm.

150. The composition according to claim 141, wherein the oily phase is present in an amount ranging from 0.5% to 40% by weight, relative to the total weight of the emulsion.

151. The composition according to claim 141, wherein the oily phase comprises at least one oil chosen from oils of animal or plant origin, mineral oils, synthetic oils, silicone oils and aliphatic hydrocarbons.

152. The composition according to claim 141, further comprising at least one other fatty substance chosen from fatty alcohols, fatty acids, waxes and gums.

153. The composition according to claim 152, wherein the at least one other fatty substance is present in an amount ranging from 2% to 10% by weight, relative to the total weight of the emulsion.

154. The composition according to claim 152, wherein the at least one fatty alcohol is chosen from stearyl alcohol, cetyl alcohol and behenyl alcohol, and wherein the at least one fatty acid is chosen from stearic acid, palmitic acid and behenic acid.

155. The composition according to claim 154, wherein the at least one fatty alcohol is cetyl alcohol.

156. The composition according to claim 152, wherein the at least one other fatty substance is chosen from glyceryl mono-, di- or tristearate and glyceryl mono-, di- or tripalmitate.

157. The composition according to claim 141, wherein the solid particles are chosen from pigments, fillers, and mixtures thereof.

158. The composition according to claim 141, wherein the dispersed solid particles are present in an amount ranging from 1% to 25% by weight, relative to the total weight of the composition.

159. The composition according to claim 141, further comprising at least one adjuvant chosen from antioxidants, fragrances, preserving agents, neutralizers, waxes, sunscreens, vitamins, moisturizers, self-tanning compounds and antiwrinkle active agents.

160. The composition according to claim 141, wherein it is in a form chosen from a foundation, a lip makeup product, an eyeshadow, a makeup rouge, a concealer product, a body makeup product, an eyeliner, and a mascara.

161. A makeup composition comprising:
(i) an emulsion comprising an oily phase dispersed in an aqueous phase, and
(ii) solid particles dispersed in the aqueous phase of the emulsion such that the makeup composition is stable after storage for two months at a temperature less than or equal to 45° C., and
wherein the emulsion comprises a surfactant system comprising:
at least one nonionic surfactant comprising at least one ethoxylated fatty ester comprising from 10 to 80 ethylene oxide units and at least one fatty acid ester of sorbitan, and
at least one ionic surfactant with a dynamic interface tension of less than or equal to 7 millinewtons/meter.

162. The composition according to claim 161, wherein the at least one nonionic surfactant is present in an amount ranging from 1% to 10% by weight, relative to the total weight of the composition.

163. The composition according to claim 161, wherein the at least one ionic surfactant of the surfactant system is chosen from alkali metal salts of cetyl phosphate and alkali metal salts of palmitoyl sarcosinate.

164. The composition according to claim 163, wherein the at least one ionic surfactant of the surfactant system is chosen from potassium cetyl phosphate and sodium palmitoyl sarcosinate.

165. The composition according to claim 161, wherein the weight ratio of the at least one ionic surfactant to the at least one nonionic surfactant ranges from 0.02 to 75.

166. The composition according to claim 161, wherein the weight ratio of the ethoxylated fatty ester to the fatty acid ester of sorbitan ranges from 0.02 to 100.

167. The composition according to claim 161, wherein the fatty acid ester of sorbitan is present in an amount ranging from 0.1% to 10% by weight, relative to the total weight of the composition.

168. The composition according to claim 161, wherein the ethoxylated fatty ester is present in an amount ranging from 0.01% to 10% by weight, relative to the total weight of the composition.

169. The composition according to claim 161, wherein the at least one ionic surfactant is present in an amount ranging from 0.05% to 10% by weight, relative to the total weight of the composition.

170. The composition according to claim 161, wherein the emulsion comprises oil globules with a number-average size of less than or equal to 500 nm.

171. The composition according to claim 161, wherein the oily phase is present in an amount ranging from 0.5% to 40% by weight, relative to the total weight of the emulsion.

172. The composition according to claim 161, wherein the oily phase comprises at least one oil chosen from oils of animal or plant origin, mineral oils, synthetic oils, silicone oils and aliphatic hydrocarbons.

173. The composition according to claim 161, further comprising at least one other fatty substance chosen from fatty alcohols, fatty acids, waxes and gums.

174. The composition according to claim 173, wherein the at least one other fatty substance is present in an amount ranging from 2% to 10% by weight, relative to the total weight of the emulsion.

175. The composition according to claim 173, wherein the at least one fatty alcohol is chosen from stearyl alcohol, cetyl alcohol and behenyl alcohol, and wherein the at least one fatty acid is chosen from stearic acid, palmitic acid and behenic acid.

176. The composition according to claim 173, wherein the at least one fatty alcohol is cetyl alcohol.

177. The composition according to claim 175, wherein the at least one other fatty substance is chosen from glyceryl mono-, di- or tristearate and glyceryl mono-, di- or tripalmitate.

178. The composition according to claim 161, wherein the solid particles are chosen from pigments, fillers, and mixtures thereof.

179. The composition according to claim 161, wherein the dispersed solid particles are present in an amount ranging from 1% to 25% by weight, relative to the total weight of the composition.

180. The composition according to claim 161, further comprising at least one nonionic aqueous thickener.

181. The composition according to claim 161, further comprising at least one adjuvant chosen from antioxidants, fragrances, preserving agents, neutralizers, waxes, sunscreens, vitamins, moisturizers, self-tanning compounds and antiwrinkle active agents.

182. The composition according to claim 161, wherein it is in a form chosen from a foundation, a lip makeup product, an eyeshadow, a makeup rouge, a concealer product, a body makeup product, an eyeliner, and a mascara.

183. A makeup composition comprising:
(i) an emulsion comprising an oily phase dispersed in an aqueous phase, and
(ii) solid particles having a number-average size of less than or equal to 10 μm dispersed in the aqueous phase of the emulsion,
wherein the emulsion comprises a surfactant system comprising:
at least one nonionic surfactant comprising at least one ethoxylated fatty ester comprising from 10 to 80 ethylene oxide units and at least one fatty acid ester of sorbitan, and
at least one ionic surfactant with a dynamic interface tension of less than or equal to 7 millinewtons/meter.

184. The composition according to claim 183, wherein the solid particles have a number-average size ranging from 0.5 μm to 10 μm.

185. The composition according to claim 183, wherein the at least one nonionic surfactant is present in an amount ranging from 1% to 10% by weight, relative to the total weight of the composition.

186. The composition according to claim 183, wherein the at least one ionic surfactant of the surfactant system is chosen from alkali metal salts of cetyl phosphate and alkali metal salts of palmitoyl sarcosinate.

187. The composition according to claim 186, wherein the at least one ionic surfactant of the surfactant system is chosen from potassium cetyl phosphate and sodium palmitoyl sarcosinate.

188. The composition according to claim 183, wherein the weight ratio of the at least one ionic surfactant to the at least one nonionic surfactant ranges from 0.02 to 75.

189. The composition according to claim 183, wherein the weight ratio of the ethoxylated fatty ester to the fatty acid ester of sorbitan ranges from 0.02 to 100.

190. The composition according to claim 183, wherein the fatty acid ester of sorbitan is present in an amount ranging from 0.1% to 10% by weight, relative to the total weight of the composition.

191. The composition according to claim 183, wherein the ethoxylated fatty ester is present in an amount ranging from 0.01% to 10% by weight, relative to the total weight of the composition.

192. The composition according to claim 183, wherein the at least one ionic surfactant is present in an amount ranging from 0.05% to 10% by weight, relative to the total weight of the composition.

193. The composition according to claim 183, wherein the emulsion comprises oil globules with a number-average size of less than or equal to 500 nm.

194. The composition according to claim 183, wherein the oily phase is present in an amount ranging from 0.5% to 40% by weight, relative to the total weight of the emulsion.

195. The composition according to claim 183, wherein the oily phase comprises at least one oil chosen from oils of animal or plant origin, mineral oils, synthetic oils, silicone oils and aliphatic hydrocarbons.

196. The composition according to claim 183, further comprising at least one other fatty substance chosen from fatty alcohols, fatty acids, waxes and gums.

197. The composition according to claim 196, wherein the at least one other fatty substance is present in an amount ranging from 2% to 10% by weight, relative to the total weight of the emulsion.

198. The composition according to claim 196, wherein the at least one fatty alcohol is chosen from stearyl alcohol, cetyl alcohol and behenyl alcohol, and wherein the at least one fatty acid is chosen from stearic acid, palmitic acid and behenic acid.

199. The composition according to claim 198, wherein the at least one fatty alcohol is cetyl alcohol.

200. The composition according to claim 196, wherein the at least one other fatty substance is chosen from glyceryl mono-, di- or tristearate and glyceryl mono-, di- or tripalmitate.

201. The composition according to claim 183, wherein the solid particles are chosen from pigments, fillers, and mixtures thereof.

202. The composition according to claim 183, wherein the dispersed solid particles are present in an amount ranging from 1% to 25% by weight, relative to the total weight of the composition.

203. The composition according to claim 183, further comprising at least one nonionic aqueous thickener.

204. The composition according to claim 183, further comprising at least one adjuvant chosen from antioxidants, fragrances, preserving agents, neutralizers, waxes, sunscreens, vitamins, moisturizers, self-tanning compounds and antiwrinkle active agents.

205. The composition according to claim 183, wherein it is in a form chosen from a foundation, a lip makeup product, an eyeshadow, a makeup rouge, a concealer product, a body makeup product, an eyeliner, and a mascara.

206. A cosmetic process for making up keratin materials, comprising applying to the keratin materials a makeup composition comprising:
(i) an emulsion comprising an oily phase dispersed in an aqueous phase and
(ii) solid particles dispersed in the aqueous phase of the emulsion such that the makeup composition is stable after storage for two months at 45° C.,
wherein the emulsion comprises a surfactant system comprising:
at least one nonionic surfactant, and
at least one ionic surfactant with a dynamic interface tension of less than or equal to 7 millinewtons/meter.

207. A cosmetic process for making up keratin materials, comprising applying to the keratin materials a makeup composition comprising:
(i) an emulsion comprising an oily phase dispersed in an aqueous phase, and
(ii) solid particles having a number-average size of less than or equal to 10 μm dispersed in the aqueous phase of the emulsion,
wherein the emulsion comprises a surfactant system comprising:
at least one nonionic surfactant, and
at least one ionic surfactant with a dynamic interface tension of less than or equal to 7 millinewtons/meter.

208. A process for preparing a makeup composition, comprising:
preparing an emulsion by mixing an aqueous phase and an oily phase with vigorous stirring, at a temperature ranging from 60 to 95° C., and then in homogenizing the mixture at a pressure ranging from 6×10⁷ Pa to 18×10⁷ Pa;
separately preparing a pigmentary paste, by dispersing solid particles in an aqueous phase in the presence of at least one ionic surfactant with a dynamic interface tension of less than 7 millinewtons/meter; and
introducing the pigmentary paste into the emulsion with moderate stirring, conserving the structure of the emulsion of the composition.
